(19) United States
(12) Patent Application Publication
(10) Pub. No.: US 2009/0192619 A1
(43) Pub. Date: Jul. 30, 2009

(54) Passive electro-magnetically damped joint

(75) Inventors: James Jay Martin, Oklahoma City, OK (US); Joel Schulz, Edmond, OK (US); Pravin Chaubey, Oklahoma City, OK (US); Craig Haslam, Oklahoma City, OK (US)

Correspondence Address: Phillips Murrah, P.C., 101 N. Robinson Ave., Corporate Tower, 13th Floor, OKLAHOMA CITY, OK, 73102, US

(73) Assignee: ORTHOCARE INNOVATIONS LLC

(21) Appl. No.: 12/313,620

(22) Filed: Nov. 21, 2008

Related U.S. Application Data
(60) Provisional application No. 61/004,144, filed on Nov. 23, 2007.

(51) Int. Cl. A61F 2/30; A61F 2/48

(52) U.S. Cl. 623/18.11; 623/24

(57) ABSTRACT

The present invention comprises an apparatus, system and method utilizing a passive electro-magnetically damped joint for prosthetics or orthotics. Such as system may be controlled through changing the resistive nature of the circuit in which a braking or damping mechanism can sufficiently replicate and augment biomechanical movement. This may be accomplished through electronic circuitry means only, or through the use of intelligent control through a microprocessor and dynamic ambulation replication algorithms.

### CROSS-REFERENCE TO RELATED APPLICATIONS

Priority is claimed from provisional patent application U.S. Ser. No. 61/004,144, filed on Nov. 23, 2007, and incorporated by reference herein.

### 1. FIELD OF THE INVENTION

The present invention relates generally to prosthetics and orthotics. More particularly, the present invention is a new and improved passive electro-magnetically damped joint.

### 2. DESCRIPTION OF THE KNOWN PRIOR ART

In the field of prosthetics, there remains a limited ability to control prosthetics and orthotics joints in a suitable manner for practical clinical application. While many advances are taking place in the field to allow for better prosthetics and orthotics joints that include adaptive control, these systems are often heavy, bulky, expensive, and require significant battery power. There remains a need for better prosthetics joints that minimize these challenging design aspects.

Conventional approaches for computer enhanced, or computer controlled prosthetic or orthotic joint systems typically use sensors, microprocessor, actuator, and battery, all configured in a complete circuitry to allow the system to move in an appropriate manner, in conjunction with the users biomechanics. This complete circuit, or series of circuits, provides a system capable of effective ambulation for an orthotic or prosthetic wearer.

Prosthetic joints currently found in the market include Magnetorheological Fluid based actuator joint of the Ossur Rheo Knee, Hydraulic based actuator joint of the Otto Bock C-leg, Pneumatic based actuator joint of the Endolite Knee, and Electro-mechanical based joint of the Touch Bionics i-Limb.

Orthotic based computer controlled joints are in their infancy on the commercial market, but similar benefits can be found in these computer controlled/enhanced devices in the literature and research labs as in the prosthetics counterparts.

Each of these methods of joint actuation requires significant power consumption to function. Because these conventional systems use actuators and components that require electric power, batteries are necessary. With conventional battery technology, this adds significant weight and size to the system. Further, because batteries or other electric power storage devices have a limitation in their capacity, there remains a limitation of usable usage time of the system. This proves to be a limitation in the functional abilities of the orthotic or prosthetic wearer, as charging capabilities are not always accessible. The user must have access to charging methods at certain incremental periods of time, such as every couple days to recharge the system. While this may not be outside of practicality for many individuals, taking a camping trip for instance may not be suitable for an individual using one of these systems.

Certain efforts have been undertaken to provide harvesting of energy for these systems, allowing for the ambulation activities to result continued or incremental charging of the system. See Donelan, Pub No. WO/2007/016781. One challenge that remains with this approach is that the circuit and actuators typically require significantly more power than what energy harvesting devices can offer. While self-charging systems may provide longer usage between charges, they do not limit the need for re-charging altogether.

In Donelan, energy is harvested across a joint, in a specified manner as to work on conjunction or mutualistic conditions with the anatomical or prosthetic joint to extract energy. This mechanical damping is converted to electrical energy which is used, in whole or in part, to power electrical components of a prosthesis, or other outside electrical components.

The energy harvester apparatus in Donelan, is selectively engaged to optimize energy harvesting while the user is in dynamic motion. Feedback loop as depicted in the application fails to conceptualize the need for fully assessing the biomechanics of amputee gait, and relies on determining when mutualistic conditions are present to gain energy harvesting from the apparatus, which would not induce increased energy expenditure of the user while the actuator is engaged. These mutualistic conditions require the use of a microprocessor to determine when to engage or disengage the energy harvesting device in order to optimize energy efficiency.

The disclosed invention described below does not require the use of a microprocessor, and optimizes the biomechanical function of user's ambulation, not optimizing the energy harvesting. Further, the disclosed system does not require the use of energy harvesting to control the function of the system, with or without the use of a microprocessor.

The prior art further fails to embody the inductive brake in a suitable package for clinical prosthetics applications. The method of packaging the device requires unreasonably large size, and inherently limits the durability and noise abatement potential of the design. The utility of the Donelan patent is purposed as an energy harvesting apparatus, and therefore inherently has a differing set of usability requirements than is necessary in clinical prosthetics applications.

Further, the energy harvester mechanism described in Donelan is tailored to the capture of energy during ambulation, for the purpose of providing power charging to other devices, and does not allow the capabilities of broad joint damping requirements.

To control a prosthetic or orthotic joint, to work in practical union with the anatomical biomechanics, a large force gradient is required. A typical trans-femoral amputee for instance ambulating on a damped knee joint can load incredibly significant amounts of torque on the device during ambulatory activities. The requirement for a joint to be able to have free range of motion movement, as would be found while the prosthetic device would be in mid-swing, is essential for ambulation. Similarly, while the prosthesis is supporting the load of the user, while walking down a hill for instance, it must prevent excessive knee flexion, and can result in over excessive torque/load to be supported by the device.

Having this large range of force transition between the loaded and unloaded states requires unique design. Mechanical embodiments described in Donelan do not enable for this large range of force damping to occur, and are therefore not suitable for direct control of damped joints in prosthetics or orthotics applications.

In the field of prosthetics and orthotics, there remains a need for controllable joint systems that can provide a suitable range of resistance, while maintaining minimal power consumption. In particular the prior art fails to provide controllable prosthetic or orthotic joints that are adaptive in their angle and angular resistance change that are lightweight, small, has an inherently high center of mass, and cosmetic. Furthermore, the prior art fails to provide a prosthetics or orthotic joint that is inexpensive, is extremely battery efficient, and or does not require battery power at all. Still further the prior art fails to provide a robotic, animatronic, equipment or similar joint that has similar objectives as for use in prosthetics and orthotics.

Although prosthetic technology has advanced in recent years, the prior art still has failed to bridge the gap between man made prosthetics and user demand and needs. Therefore, an extensive opportunity for design advancements and innovation remains where the prior art fails or is deficient.

### SUMMARY OF THE INVENTION

In general, the present invention is a new and improved prosthetic and or orthotic joint system which provides an electro-magnetically damping action where the prior art fails. The present invention generally provides a new and improved design for actively and intelligently controlling the movement—angle and resistance of angular change—of a device to enable for improved ambulation of a prosthetics or orthotics user, while requiring minimal power consumption to do so.

In this respect, before explaining at least one embodiment of the invention in detail, it is to be understood that the invention is not limited in this application to the details of construction and to the arrangement of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods and systems for carrying out the several purposes of the present invention. It is important, therefore that the claims be regarded as including such equivalent constructions insofar as they do not depart from the spirit and scope of the present invention.

Accordingly, titles, headings, chapters name, classifications and overall segmentation of the application in general should not be construed as limiting. Such are provided for overall readability and not necessarily as literally defining text or material associated therewith. For explanatory purposes the terms “prosthetics” and “orthotics” may be used synonymously in relation to the discussion of the benefits to either or both.

Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

It is therefore an object of the present invention to provide a new and improved prosthetic or orthotic joint system that is adaptive in its angle and angular resistance change.

It is a further object of the present invention to provide a new and improved prosthetic joint system which is a relatively simple but robust and thus may be easily and efficiently manufactured.

An even further object of the present invention is to provide a new and improved prosthetic or orthotic joint system which is of a more durable and reliable construction than that of the existing known art.

Still another object to the present invention to provide a new and improved prosthetic or orthotic joint system which is susceptible of a low cost of manufacture with regard to both materials and labor, which accordingly is then susceptible of low prices of sale to the consuming public, thereby making such economically available to those in need of such prosthetic or orthotic devices.

Another object of the present invention is to provide a new and improved prosthetic or orthotic joint system which provides some of the advantages of the prior art, while simultaneously overcoming some of the disadvantages normally associated therewith.

Yet another object of the present invention to provide a new and improved prosthetic or orthotic joint system that is relatively lightweight, relatively small, and may have an inherently high center of mass.

Still yet another object of the present invention is to provide a new and improved prosthetic or orthotic joint system that is extremely battery efficient or that does not require battery power at all, while being adaptive to the ambulatory requirements.

A further object of the present invention is to provide a new and improved prosthetic or orthotic joint system which provides improved cosmetic appearance.

Still another object of the present invention is to provide a new and improved prosthetic or orthotic joint system which provides a robotic, animatronic, equipment or similar joint that has similar objectives as for use in prosthetics and orthotics.

Another object of the present invention is to provide a new and improved prosthetic or orthotic joint system which provides a mechanical utility that simulates or more closely simulates a natural human motion and function.

An even further object of the present invention is to provide a new and improved prosthetic or orthotic joint system which may simplify a users training and rehabilitation to a new prosthetic or orthotic.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference would be had to the accompanying drawings and descriptive matter in which there are illustrated preferred embodiments of the invention.

### BRIEF DESCRIPTION OF THE PICTORIAL ILLUSTRATIONS GRAPHS, DRAWINGS, AND APPENDICES

The invention will be better understood and objects other than those set forth above will become apparent when consideration is given to the following detailed description thereof. Such description makes reference to the annexed pictorial illustrations, graphs, drawings, and appendices.

FIG. 1 generally illustrates a trans-tibial lower extremity prosthesis utilizing a general embodiment of the invention.

FIG. 2 generally illustrates a trans-femoral lower extremity prosthesis utilizing a general embodiment of the invention.

FIG. 3 generally illustrates hip disarticulation lower extremity prosthesis utilizing a general embodiment of the invention.

FIG. 4 generally illustrates a motor or other inductive source that can be affected by Lenz's Law.

FIG. 5 generally illustrates a motor or other inductive source that can be affected by Lenz's Law, along with a gearing type of mechanism.

FIG. 6 generally illustrates in a preferred embodiment of the invention how the gearing mechanism amplifies the movement between two limb sections into increased motion for inducing Lenz's Law for inductive braking.

FIG. 7 generally illustrates the use of variable turn windings within the device.

FIG. 8 generally illustrates permanent magnet, electrical/mechanical controller, and winding or transformer within one embodiment.

FIG. 9 generally illustrates the orientation of the limb sections about a limb joint, along with subcomponents.

FIG. 10 generally illustrates a preferred embodiment of the invention in conjunction with additional features.

FIG. 11 generally illustrates a preferred embodiment of the invention in conjunction with additional features.

FIG. 12 generally illustrates the use of multiple motors and gearing mechanisms linked in conjunction with one another.

FIG. 13 generally illustrates a link of various motors and or gear mechanisms as generally shown.

FIG. 14 generally illustrates use of a one way clutch mechanism.

FIG. 15 generally illustrates the use of one-way clutch mechanism to further divide the different motions into two or more inductive braking mechanisms.

FIG. 16 generally illustrates a preferred embodiment of the invention incorporating gears and motor within inner and outer cylinders, and their general interaction and orientation with one another.

FIGS. 17 A and B generally illustrate a preferred embodiment of the invention incorporating gears and motor within inner and outer cylinders, and their general interaction and orientation with one another.

FIGS. 18 A, B, and C generally illustrate the relationship between joint position and experienced torque on the device, along with altering the resistance of the device according to the torque moment on the device.

FIG. 19A generally illustrates a preferred embodiment of the invention using sensor switch mechanism directly linked to induce inductive brake.

FIG. 19B generally illustrates a preferred embodiment of the invention whereas microprocessor controls movement of the device through sensor information, which may or may not use battery that is charged by the inductive brake.

FIG. 19C generally illustrates a preferred embodiment of the invention whereas microprocessor controls movement of the device through sensor information, which may include neural integration approaches.

FIG. 20A generally illustrates the stance phase of the gait cycle as is being replicated through the use of an inductive brake.

FIG. 20B generally illustrates the swing phase of the gait cycle as is being replicated through the use of an inductive brake.

FIG. 21 generally illustrates the resistive and powered actuation strategies of the inductive brake during the gait cycle for knee and ankle joints.

FIG. 22 generally illustrates a preferred embodiment of the invention using various methods of inducing inductive braking in the device.

FIG. 23 generally illustrates an ankle range of motion and torque experienced at the anatomical ankle, which is being replicated through the control strategy of the inductive brake.

FIG. 24 generally illustrates a knee range of motion and torque experienced at the anatomical ankle, which is being replicated through the control strategy of the inductive brake.

FIG. 25 generally illustrates a hip range of motion and torque experienced at the anatomical ankle, which is being replicated through the control strategy of the inductive brake.

FIG. 26 generally illustrates a preferred embodiment of the invention using inductive braking for AC and DC motor designs.

FIG. 27 generally illustrates a preferred embodiment of the invention using energy storage and delivery through a capacitor.

FIG. 28 generally illustrates a preferred embodiment of the invention transferring stored energy to power a microprocessor and/or other electronics.

FIG. 29 generally illustrates a preferred embodiment of the invention transferring stored energy to supply partial power to a microprocessor and/or other electronics.

FIG. 30 generally illustrates Lenz's Law of inductive braking.

### DETAILED DESCRIPTION OF PREFERRED EMBODIMENTS

In a preferred embodiment, the current invention may include the following although it is contemplated that combinations may be utilized to provide a electro-magnetically damped joint design, apparatus, method, and so forth as generally referred to in the application and illustrations described below. It is further contemplated the joint or joint system may be passive, non passive or combinations thereof.

The current invention joint or joint system may be used as a joint in any type of lower or upper extremity external prosthesis or orthosis (Partial Foot, Symes, Below Knee, Knee Disarticulation, Above Knee, Hip Disarticulation, Hemi-Pelvectomy, Ankle Foot Orthosis—AFO, Knee Orthois—KO, Ankle Foot Knee Orthosis AFKO, etc). The invention may be used as a forefoot joint, ankle joint, knee joint, and/or hip joint, and/or any combination of joints, including upper extremity joints—for joint replacement or joint augmentation.

It is further contemplated the current invention may also be used for any upper extremity

external prosthesis: (congenital or acquired: Partial Finger, Finger Disarticualtion, Partial Hand, Transcarpal, Wrist Disarticulation, Below Elbow, Elbow Disarticulation, Above Elbow, Shoulder Disarticulation, Four-Quarter Amputation, etc).

The invention may be utilized with or used as a finger joint, knuckle joint, wrist joint, elbow joint and/or shoulder joint, and/or any combination of joints, including lower extremity joints. The current invention may be combined with other types of joints, including the following, but not excluding any that are not mentioned, or not invented yet: friction joints, weight activated joints, pneumatic and hydraulic joints, multi-bar hinge joints, rolling joints, cam joints, powered joints and/or any combination of joint.

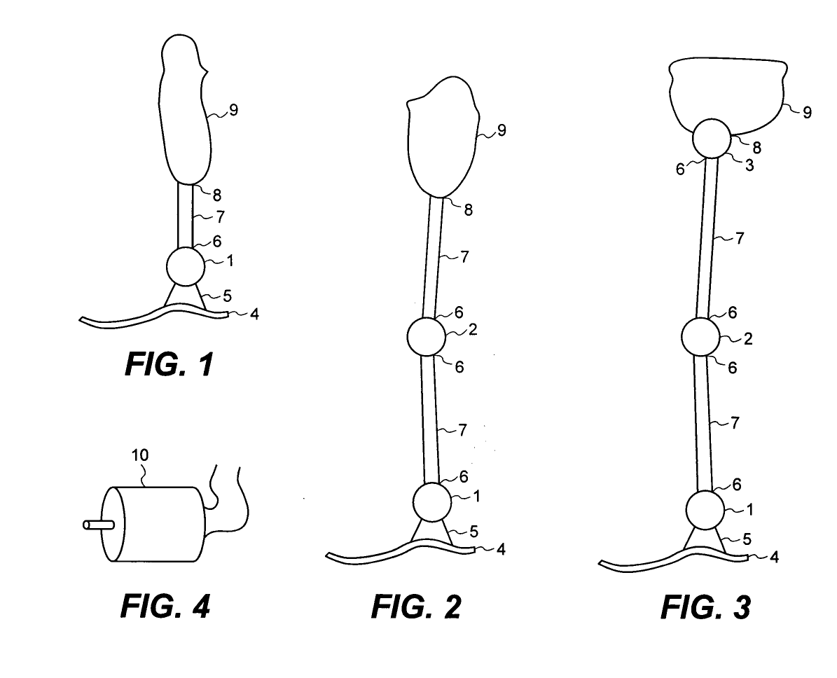

In accordance with a preferred embodiment of the invention, FIG. 1 generally depicts a below knee prosthesis and prosthetic ankle joint 1. FIG. 2 generally depicts an above knee prosthesis, knee joint 2, and/or an ankle joint 1. FIG. 3 generally depicts a hip disarticulation prosthesis, hip joint 3, and/or knee joint 2 and/or ankle joint 1. As is well understood in the field of prosthetics, these devices generally include a prosthetic foot member 4, attachment means 5 to the foot, attachment means to the pylon or shaft 6, pylon or shaft 7, attachment means to the socket interface 8, and socket interface 9.

It is equally contemplated that FIGS. 1, 2, and 3 could represent orthotic devices as well, for explanatory purposes. Joints 1, 2, and 3 in FIGS. 1, 2, and 3 may generally be configured as orthotic joints used to augment movement, versus replace joints movement as would be found in prosthetics. Other such components such as the pylon 7 would be replaced with shaft sections suitable for orthotic applications. Socket interface sections 9 would be replaced with orthotic equivalent interfaces, and prosthetic foot 4 would be replaced with orthotic equivalent brace to form device in conjunction with the anatomical counterpart.

It is understand that the general position of the joints relative to their anatomical counterpart are generally similar for prosthetics or orthotics applications. In orthotic applications, the illustrated joints would coincide in parallel with the anatomical counterpart, whereas in prosthetics, the joint simply replaces the anatomical joint. While lower extremity examples have been pictorially described in FIGS. 1, 2 and 3, it is contemplated that the upper extremity equivalent may function in similar relation to upper extremity joints for orthotic or prosthetic applications.

In typical prosthetics and orthotic applications, the use of braking or damping mechanisms can sufficiently replicate and augment biomechanical movement. During typical ambulation, many of the actions of the limbs are resistive in nature—eccentric muscular contractions. For conventional prosthetics for instance, joints may use resistive actuation means to adequately replicate biomimetic movement for most ambulatory activities. Certain activities such as walking up stairs step over step requires power input to a knee prosthetic system for instance, in order to fully replicate the anatomical counterpart. Incorporating active powered actuation into a prosthetic device may add significant complexity and weight, and while ultimately powered actuation is more complete representation of the anatomical counterpart, the disadvantages often outweigh the advantages for many prosthetics or orthotics users with today's technology. It is well understood in the field of prosthetics and orthotics how to replicate biomechanical movement through resistive actuation methods.

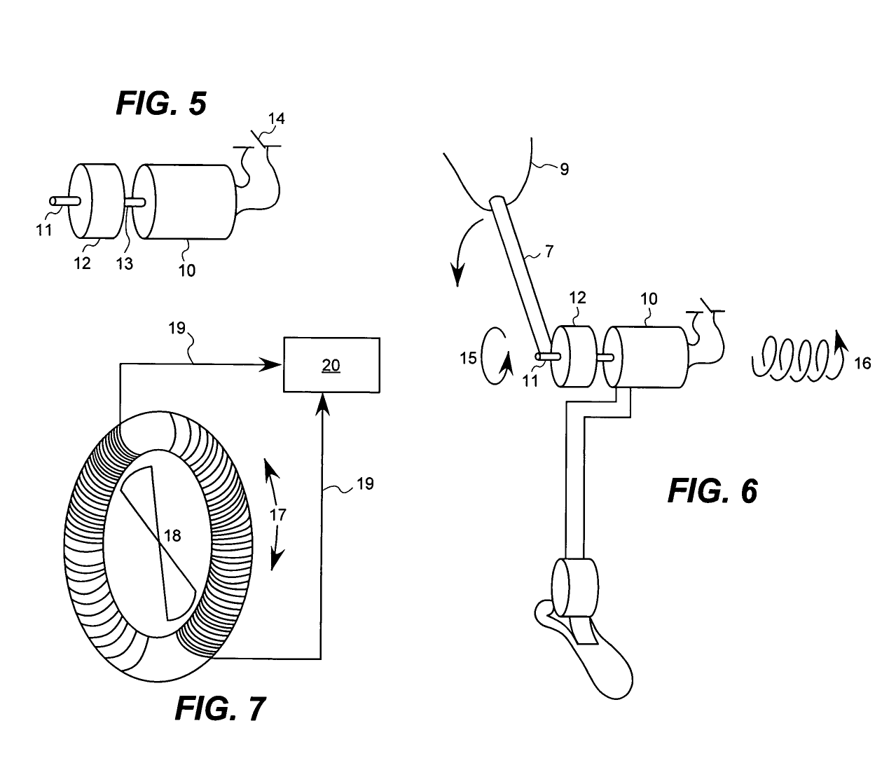

Now generally referring to FIGS. 4, 26, and 5 and the other illustrations, it is contemplated that the current invention may include a braking mechanism of the joint (possibly separate from any other mechanism in the joint, such as, but not limited to a multi-bar linkage, extension assist spring, pneumatics and/or hydraulics, powered actuation) that may comprise of the following described below. As is generally illustrated in FIG. 5, the gear 12, motor mechanism 10, and shorting mechanism 14 together, along with other device components, such as but not limited to electronics and other members described further below, may be used collectively in a controllable manner in order to initiate and sustain necessary angular and resistive changes within the joint. While the method of controlling these will be discussed in sections below, the mechanical embodiment (the controlled member) may be illustrated in any number of ways to achieve the desired effect as is described.

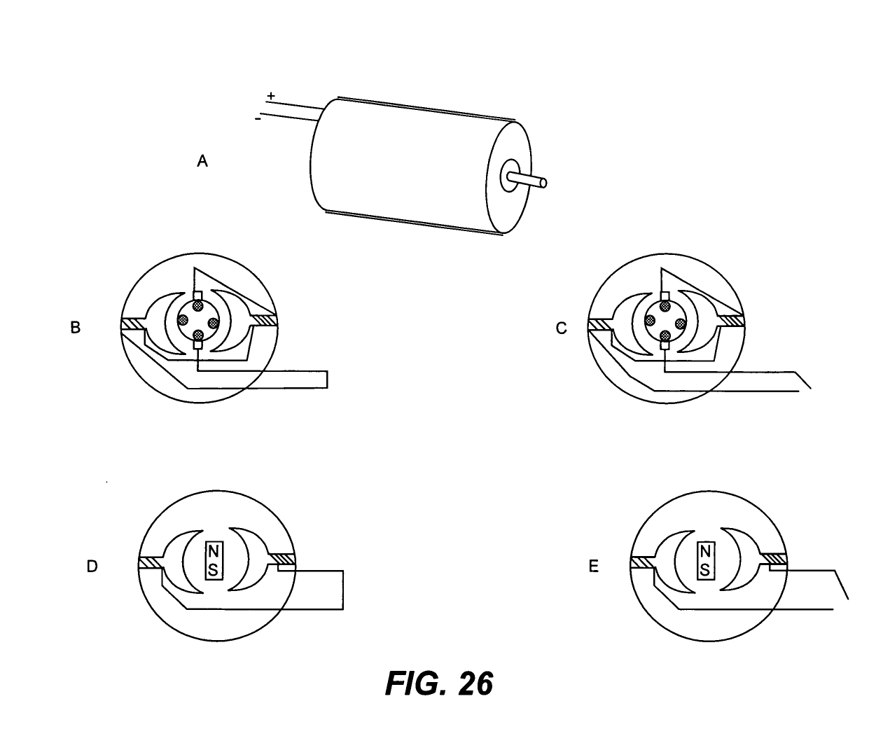

Now generally referring to FIG. 26, and other illustrations, it is contemplated that various types of mechanisms may be used for purposes of inductive braking, including but not limited to AC and DC motors. FIG. 26B illustrates an AC motor with the magnetic field in stator, with resistive force, as the circuit is closed. FIG. 26C illustrates a DC motor with no magnetic field in stator, with no resistive force, as the circuit is open. FIG. 26D illustrates a DC motor with magnetic field in stator, with resistive force, as circuit is closed. FIG. 26E illustrates a DC motor with no magnetic field in stator, with no resistive force, as circuit is open.

According to Faraday's law, any change in the magnetic environment of a wire coil will cause an electromotive force, or voltage, to be induced in the coil. The change in magnetic environment may be produced by any number of methods including changing the magnetic field strength of a magnet in proximity to the coil, moving a magnet closer to or further from the coil, moving the coil into or out of a magnetic field, or rotating a coil relative to a magnetic field, amongst others.

Lenz's law provides the direction of the induced EMF, or electromotive force, and current resulting from electromagnetic induction. This law determines the choice of sign in Faraday's law of induction, determining that the induced EMF and the change in flux have opposite signs. As a result, the current associated with the EMF will be such that the flux created will oppose the change in flux that created it. The induced EMF in a coil is equal to the negative of the rate of change of magnetic flux times the number of turns in the coil.

Faraday's law of induction describes that the EMF in any closed circuit is equal to the time rate of change of the magnetic flux through the circuit. FIG. 30 quantitatively illustrates Faraday's law of induction. The negative sign in the formula in FIG. 30 is given by Lenz's Law. Due to the law of conservation of energy, the magnetic field of any induced current opposes the change that induces it. Because of this, passing a magnet through a closed circuit coil for instance results in the production of electric current, as well as a resistive force to move the magnet through the coil. Passing a magnet through a coil produces an EMF that acts upon the electrons within the coil that are subjected to the increasing magnetic field. As the speed of the magnet is increased, the resultant resistive force increases as well. The increasing of the area of the coil results in an increasing flux through the coil. Because of this, incorporating a system using a magnet and a coil, and inducing rapid movement between the two generates inductive braking.

In a preferred embodiment, a motor system may comprise a motor, electric generator (dynamo), or any other type of magnetic/coil device 10, capable of generating inductive braking/damping by the use of Lenz's Law. It is contemplated to provide a gear mechanism input shaft 11 that may be connected to or be in line with the orthotic or prosthetic joint. The movement of the joint may be transmitted to the gear mechanism 12. The gear mechanism may serve to amplify and/or multiply the speed of the joint movement before connecting this motion to the motor or motor system 10.

In general, the term gear, gear mechanism, or other explanations of 12 should not be considered limiting, as the member 12 illustrates any of the known methods of amplifying or generally increasing or enhancing the movement from the input shaft to the motor 10.

A linkage 13 from motor to gear mechanism may be any type of connection to transmit motion from the motor to the gear mechanism such as, but not limited to the body of the motor may be a gear in itself, while the motor shaft may remain fixed, etc. A gear mechanism 12 may be comprised of, but not limited to wheels, belts, pulleys, gears, or other linkages or drive mechanisms, as well as variable gear ratio mechanisms, such as cobot devices amongst others. A gear mechanism input shaft 11 may be, but is not limited to being a gear, belts, pulley and/or wheel or other linkage, drive, or variable ratio mechanisms. It may also be comprised of, but not limited to having a gear on the outside of the gear mechanism to transmit motion from the joint to the gear mechanism.

FIG. 6 conceptually illustrates one embodiment of magnetic coil mechanism 10 in conjunction with a gear mechanism 12 to multiply rotary torque input 15 to the magnetic coil mechanism 10 from the prosthesis or orthosis. In this illustration, the weight of the user as transferred through the socket interface 9 and pylon 7 to the input shaft 11. In this illustration and embodiment, the rotary motion is then transferred through the gear mechanism 12 to the magnetic coil mechanism. As generally illustrated, the torque input 15 is translated to a multiplied rotary motion output 16. In accordance with the principle of Lenz's Law, this magnification of motion, allows for increased resistance of the braking or damping mechanism when circuit 14 is closed.

A shorting mechanism 14 may be an electrical switch. It is contemplated that when this closes the circuit, thus ‘shorting’ the motor 10 or motor system (FIG. 5), it may create an inductive brake due to the physics principle of Lenz's Law. This may create a high resistive force to the gear mechanism input shaft 11. The shorting mechanism 14 could be, but not limited to a switch, a weight-activated switch, an electronically controlled switch, a micro-processor activated switch, a potentiometer, transistor, capacitor, resistor, battery, diode, or other mechanisms that may account for closing the circuit, etc.

It is further contemplated that this could also be, but not limited to, wired to a motor or other electronic device that draws current. This could also be, but not limited to, connected to a motor or similar actuator that drives a vacuum pump or compressor, for example a socket vacuum system, and/or a pressure accumulator, and/or a socket heating/cooling system, or may be used to store energy in a battery or capacitor.

A braking mechanism 10 which provides torque to the system may also be primary or secondary winding of a transformer moving inside a magnetic field. The magnetic field can be created using permanent magnet or electromagnets. In such a setup, the relative motion between the rotor and stator can be created by moving any one of two or both. In another implementation, a permanent magnet 18 can be surrounded by coils with increasing number of turns 17. Number of turns is proportional to damping force. It is generally desired to have a large number of turns of the coil to enhance the effects of Lenz's Law in the preferred embodiments.

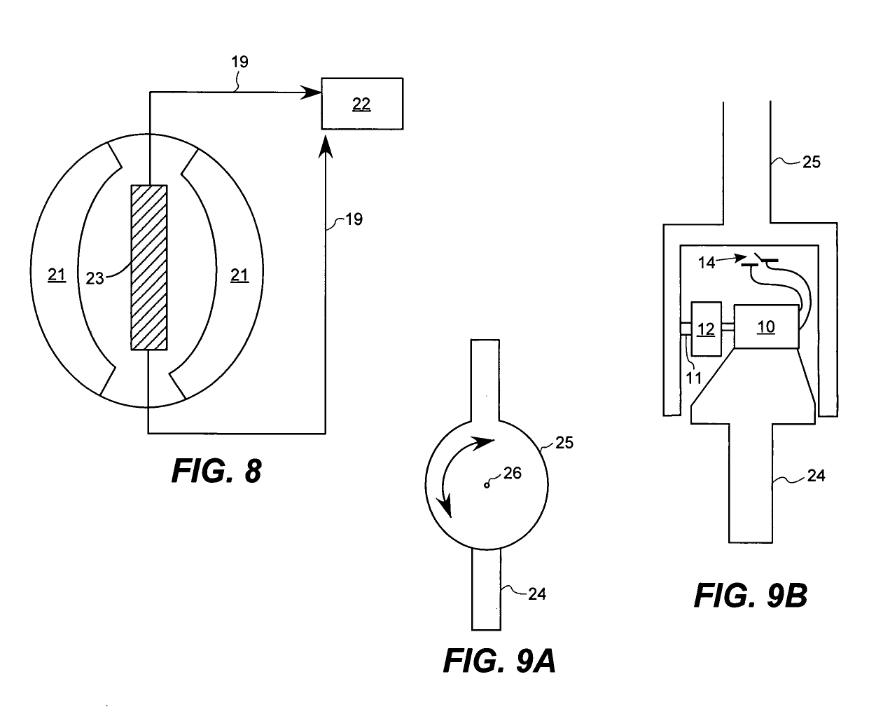

Now generally referring to FIGS. 7, 8, 9, and 10 and the other illustrations, it is contemplated that within the braking mechanism 10 to provide variable turn winding 17, permanent magnet or electromagnet 18, connectors 19, electrical/mechanical controller 20, permanent magnet 21, electrical/mechanical controller 22, winding or transformer 23.

Now generally referring to FIGS. 9A and 9B side and cut-away front views and the other illustrations, the main “joint housing” may be comprised of two pieces ‘A’ 24, and ‘B’ 25 that may comprise the basic prosthetic joint, essentially a hinge, pivot, and/or axis. Part ‘B’ of the joint 25 may transmit the relative motion between itself and the second part ‘A’ of the joint 24 to the gear mechanism input shaft 11. Part ‘A’ of the joint 24 may be fixed to the body of the “motor” 10.

The joint housing, ‘A’ 24, and ‘B’ 25, may essentially rotate relative to each other via an “axis” 26. It may not matter whether ‘A’ 24 or ‘B’ 25 is the fixed or the rotating element. It is also contemplated that the main joint housing, ‘A’ 24, and ‘B’ 25, may also carry the main loads of the joint, using, but not limited to ball bearings, roller bearings, bushings, plastic bearings, etc. There may or may not be endpoint stopping mechanisms to limit and/or dampen the total range of motion of the joint.

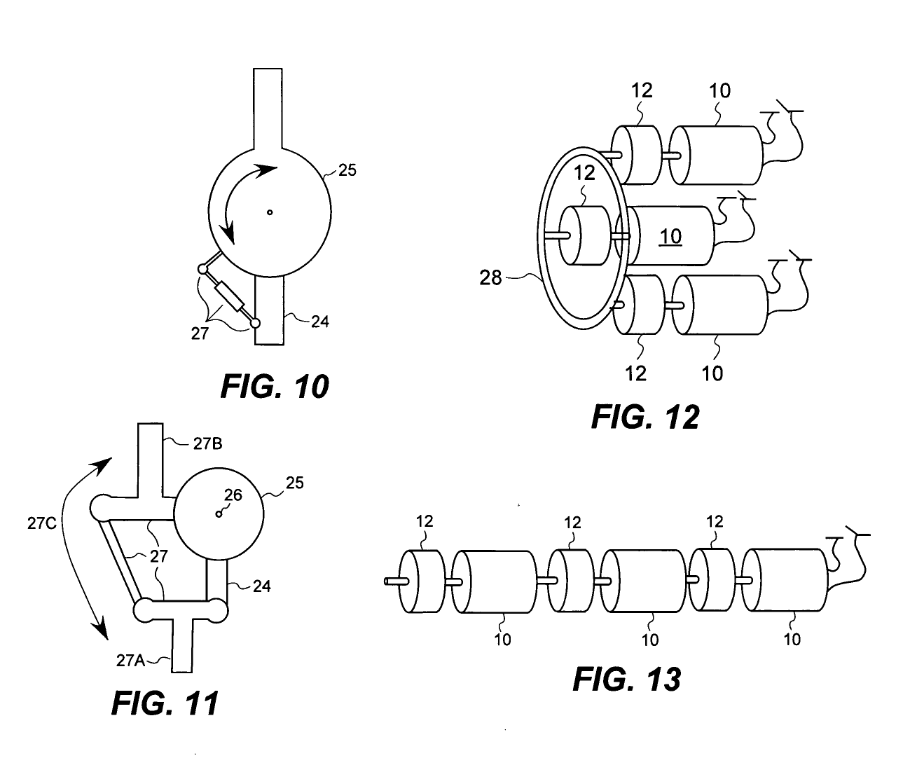

Now generally referring to FIG. 10 and other illustrations, it is contemplated to provide linkages 27 that may include, but are not limited to any one, or combination of gears, braking systems, springs, pneumatic and/or hydraulic mechanisms to further control the joint. These may be linear in nature, as generally depicted, rotary type, or other embodiments and or combinations thereof. These linkages 27 may be combined with motion generating mechanisms that include, but are not limited to any one, or combination of engines, springs, motors, pneumatic and/or hydraulic motor systems that provide powered motion to the joint. These may be linear in nature, as generally depicted, rotary type, and or combinations thereof. Linkages 27 may also be power generating in nature, such as, but are not limited to, electric generators or dynamos, inductive coils, piezoelectric generators, compressed gas or fluid generators, heat generators, etc. It is contemplated that any way of storing power to be used to drive the joint and/or electronics, etc. These may be linear in nature, as generally depicted, rotary type, and or combinations thereof.

Still furthermore, it is contemplated that the magnetic coil mechanism 10 with gears mechanism 12 along with other necessary subcomponents may be electrically powered in order to induce a positive power input to the system—essentially using the device as an electric motor system for power generation. This may be used at particular moments or durations during the gait cycle in order to power the user with positive power input as needed during ambulation, and may be used in conjunction with damping methods as found in Lenz Law.

Now generally referring to FIG. 11 and other illustrations, it is contemplated to provide a joint housing 24, 25, 26 which may be a component of a more complex prosthetic joint such as but not limited to a multi-bar linkage 27, 27A, 27B. A multi-bar linkage can use the joint housing to produce an elliptical motion 27C such as the illustrated arc-pathway of the relative motion of 27A to 27B. These may be linear in nature, as generally depicted, rotary type, and or combinations thereof. The FIG. 11 should not be considered limiting, as there are a number of known methods in the prior art, and in the field in general, to incorporate a prosthetic or orthotic joint along with mechanical implementations to include but not limited to one, two, three, four, five, six, seven, or more, axis points to create a complex linkage system for providing asymmetrical or elliptical motion of a joint. It is therefore further understood that the disclosed invention does not need to reside at or near the center of rotation of the joint itself.

It is contemplated to provide more than one joint housing in a single prosthetic joint. These multiple joint housings may be used in the same plane of motion, and/or may be used to control multi-planar motion. It is contemplated to provide, but not limited to, in an ankle joint plantarflexion-dorsiflexion, in the saggital plane, and/or inversion-eversion, in the frontal or coronal plane, and/or toe-in and toe-out, in the transverse plane.

The different linkages can also be mechanically and/or electronically switched, such as but not limited to changing the gears, to give different resistive characteristics to the prosthetic joint. The user of the device may normally desire a light resistance for typical ambulation, but may desire a higher resistance for descending a hill, etc.

Now generally referring to FIG. 12 and other illustrations, it is contemplated to provide multiple motors and or multiple gear mechanisms by mechanically linking them together 28. They may be linked with any of the following, but not limited to belts, pulleys, gears, etc. It is further contemplated to link various motors and or gear mechanisms as generally shown in FIG. 13.

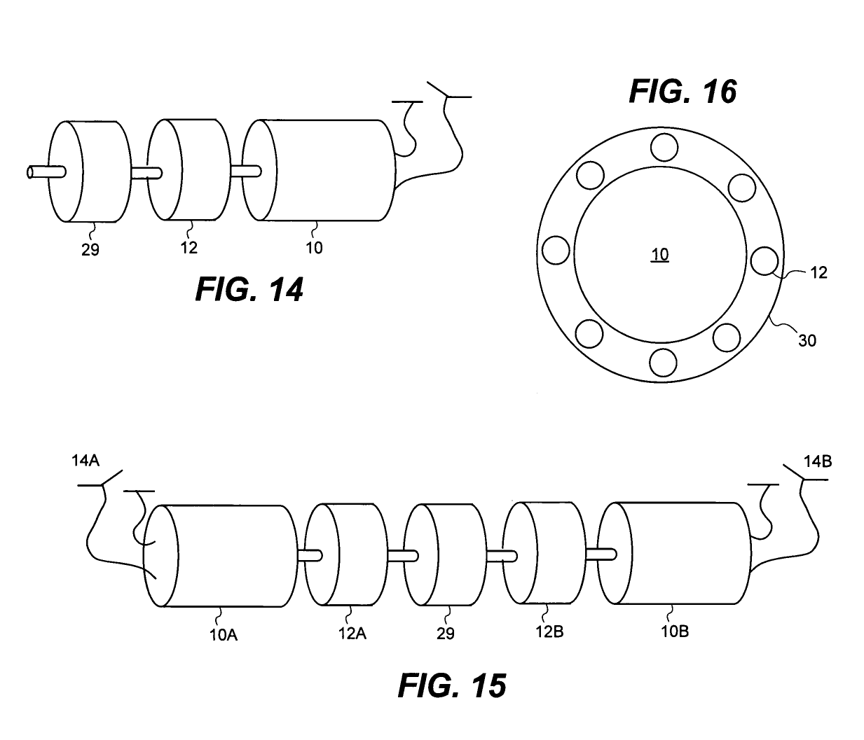

Now generally referring to FIG. 14 and other illustrations, it is contemplated to utilize a one-way clutch mechanism 29. This may be positioned anywhere in the final prosthetic joint, or attached to the gear mechanism as generally shown. This may allow one direction of the prosthetic joint movement to engage the inductive brake, but movement in the other direction may not engage the inductive joint or may be only partially engaged. Unit 29 could also be a mechanism that involves a linkage like but not limited to a differential, that can divide the input motion into two different gear-boxes, depending upon the direction, and/or speed, and/or force, and/or position of the input motion. This is contemplated because within certain prosthetic and orthotic joints, there may be a desire to dampen the movement in one but not both direction in order to provide suitable and appropriate biomechanical movement. For instance, in certain circumstances, it may not be necessary to use Lenz's Law principle to dampen swing knee extension, but only stance knee flexion. Therefore, it may be desired in certain instances to inhibit any inherent friction that may alter the swing characteristics of the joint. Therefore, disengaging the gear or other mechanism 12 during this time is desired. This disengagement of the gearing member 12 may be performed mechanically or electronically, and may utilize other outside members not shown such as solenoid, valve, motor, spring, or other mechanical, electromechanical, or fluid actuated methods.

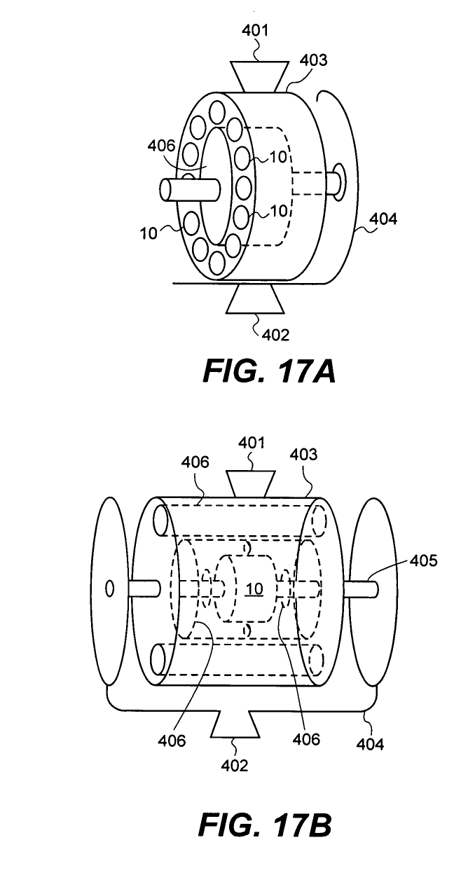

The initiation of any number of multiple gear members may be utilized to further vary the amount of torque resistance found in the device. It is further contemplated that gears 12 and motor 10 may reside in a common housing, and/or generally surrounding each other. As is generally depicted in FIGS. 16 and 17, motor 10 is surrounded by gear members 12, and together may be held within housing 30. The exact nature of the relationship between the inner motor and outer gear member may be illustrated in any number of methods, as there are many ways of amplifying motion between the inner and outer member known in the art. It is further understood that gears 12 may be surrounded by motor(s) 10, as would be the reverse of the illustrated embodiment.

The mechanical illustrations presented inherently offer proximal weight distribution within the prosthetic joint. This is especially significant as pendular effects of prosthetic or orthotic weight can alter the gait of the user. In general, the higher the weight of the device, the less pendular effects are noticed by the user. If the weight of a prosthesis is very distal, such as a heavy prosthetic foot/ankle for instance, it may result in excessive knee flexion at initial swing phase of gait, as well as limited knee extension at terminal swing due to pendular effects. While these can be somewhat mitigated by control means of the joint, their effect can have further influence on energy expenditure of the user.

Still further, embodiment illustrated in FIGS. 16 and 17 in particular may provide enhanced cosmetic appearance, as the envelope of the design may be encompassed within the anatomical profile. Having the rotary motion of the device, and the various internal mechanisms consolidated into a small package provides a device that can be easily cosmetically covered.

The cosmetic appearance of the device however is not limited to the physical embodiment. Rather, the cosmetic appearance of the device is largely noticed through the life-likeness of its movement. Having a control strategy that is capable of providing the appropriate movement in correspondence with the anatomical counterpart provides the look of natural movement, making the prosthetic or orthotic device less noticeable. The methods of incorporating dynamic cosmetic appearance in the device will be further discussed below in the control system discussion.

Now generally referring to FIG. 15 and other illustrations, it is contemplated to provide a one-way clutch mechanism 29 could also divide the different motions into two or more inductive braking mechanisms ‘A’ (10A, 12A, and 14A) and ‘B’ (10B, 12B, and 14B). This may not need to be linked as in the illustration, but may simply be a means of combining multiple inductive braking mechanisms into a joint. Each inductive braking mechanism could have its own particular linkage to different aspects of the joint, with different braking characteristics. For example, but not limited to a knee joint, knee flexion drives one inductive braking mechanism, while knee extension drives another inductive braking mechanism.

Now generally referring to FIGS. 16 and 17 and other illustrations, it is contemplated to utilize an embodiment whereas the motor and gears are generally housed within circumferentially surrounding components to optimize space considerations to fit within the anatomical envelope for more appropriate cosmetic appearance. It is contemplated that any number of gears may surround a motor, or visa versa in order to promote an increase in the amount of relative motion between the area proximal to the joint and distal to the joint. These motors 10 and gears 12 may be switched in order to provide multiple motors to be driven by a set of gears (FIG. 12), or may have any number of gearing mechanisms to drive a single motor. The motor shaft may be linked to the joint axis of rotation, and the outer housing may linked to the external prosthetic section, so that as there is motion between the proximal and distal aspects of the joint, the gears result in rotation of the motor element, causing EMF, and hence resistance in the joint. By using the housing mechanism around the gears and motor, and because the housing element may be a load bearing component, the inherent mass of that unit may limit any noise the gears and motor may cause.

The housing assembly can for purposes of explanation be split into inner an outer cylinders. The inner cylinder 403 may be affixedly connected to one attachment point 401, while the outer cylinder 404 may be affixedly connected to the other attachment point 402. The inner an outer cylinders may move in rotary or relative motion with each other, resulting in the two attachment points to move in relation to each other. The motor unit 10 may be affixedly connected to either the inner 403 or outer 404 cylinder. For purposes of explanation, the illustration depicts the motor attached to the outer cylinder by way of the shaft 405. The shaft and outer cylinder 404 are affixedly connected together. In the space between the outer 404 and inner 403 cylinders are a number of gearing mechanisms 406 that allow for a multiplication of motion between the inner 403 and outer 404 cylinders. This allows for an increase in Lenz's law, resulting in increased resistance to motion between the inner 403 and outer 404 cylinders. It is understood that the illustration is simplified for the purposes of explanation, and should not be considered limiting.

The inner 403 and outer 404 housing sections together make up a prosthetic or orthotic joint, replicating the size, position, general shape, and function of the anatomical counterpart. By being structural members, they inherently provide increased durability to the inner components, their inherent mass decreases any noise the inner components may create, and provide a high center of mass and low build height of the system.

There may as well be inner components, not shown in the figure that mechanically engage and disengage gears or similar mechanisms to enable for more free of motion when needed. There may also be multiple motors that can be engaged or disengaged, all linked to the inner and outer cylinder whereas motion between the inner and outer cylinders results in extrapolated motion of the motors, and hence resistance.

It is contemplated to provide control strategies of the shorting mechanism 14, which may be done in multiple ways, but not limited to non-powered electronic, powered electronic and/or microprocessor.

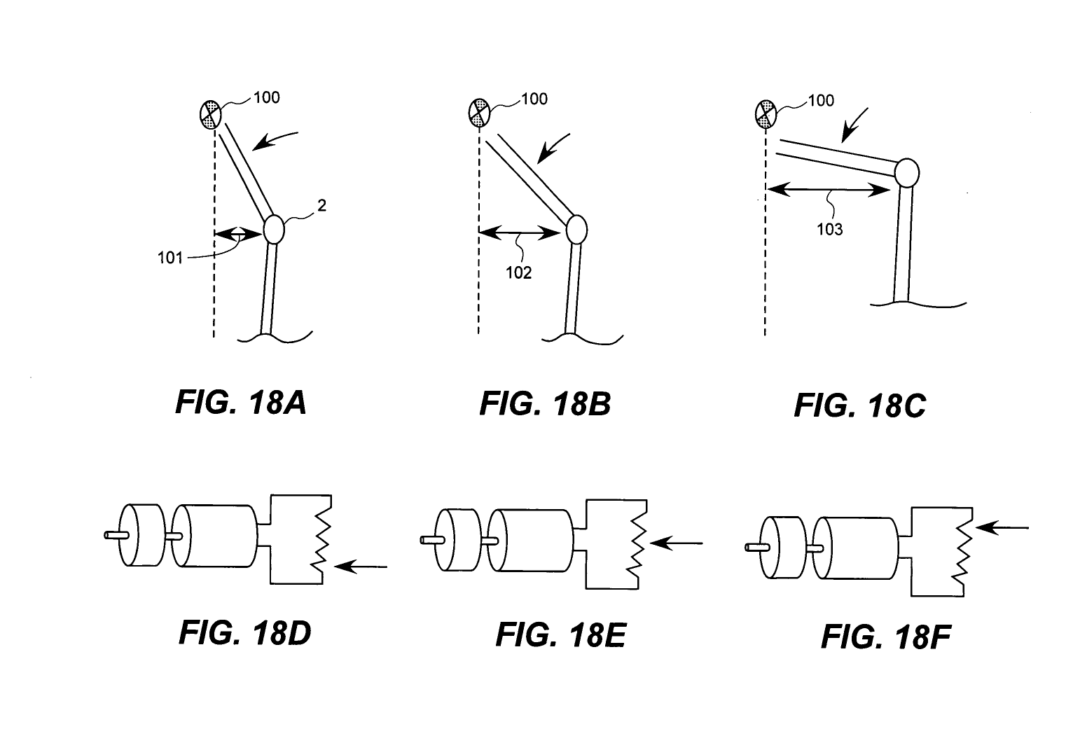

Non-powered electronic control is advantageous in that it allows for a simple and inexpensive design, which may provide suitable biomechanical replication during various activities of use. Now generally referring to FIGS. 18A, 18B, and 18C and other illustrations, a system may use a switch that a user activates, or a user's movements activate in order to cause inductive braking, such as but not limited to having high resistance in a knee as the user sits down, so the user can “ride” the knee as they sit. In such an example, a potentiometer, or similar adjustable resistance mechanism, may be used as a switching mechanism, corresponding the resistance of the inductive brake to the angle of the joint.

In such a case, the user illustrated in FIG. 18A is in a motion causing knee flexion, sitting for example. While the knee joint 2 is bending in a flexion direction, the distance between the body's center of mass 100 and the knee joint 2, 101, is increasing. FIGS. 18B and 18C show an increasing distance between the body's center of mass 100 and the knee joint 2 respectively, 102 and 103. This results in increasing torque about the knee joint. FIGS. 18D, 18E, and 18F illustrate a potentiometer increasing in resistance according to the knee angle experienced in corresponding illustrations 18A, 18B, and 18C. This results in countering the torque experienced at the knee joint, enabling for the user to lower their center of mass in a controlled manner.

The amount of inductive braking resistance experienced may be correlated with maintaining the angular velocity of the joint, maintaining angular position of the joint, maintaining constant speed, providing a varied angular velocity, angular position, speed, or torque, or using proportional control corresponding to force, speed, angular velocity, resistance, torque, acceleration, or other such sensor data.

While the example illustrated and discussed above and below may depict a knee joint, it is understood that similar relationships may be found in other joints and in other actions. The knee joint is simply used for example so that those skilled in the art may easily comprehend a method of controlling such a device.

It is further contemplated to provide, but not limited to, any number of combination of weight activated switches and or potentiometers, spring-loaded switches and or potentiometers, weighted switches and or potentiometers, etc that could produce the following described below.

In a knee for instance, a weight activated switch may produce particular or adaptive resistance inductive braking whenever the user has a certain amount of weight or torque through the joint, thus preventing the knee from buckling while the user needs it for support in standing and in stance phase of walking. A weighted potentiometer could control the resistance of the joint as the angle of the joint and/or prosthesis changes, thus increasing resistance as the user sits, since their relative force vector will increase the more the knee angle changes. A spring-loaded switch may be able to close the circuit if the angular acceleration of the joint and/or prosthesis is excessive—possibly detecting that the user may be falling and needs maximum or higher resistance form the joint. Potentiometers could be used in combination with joint linkages to increase the joint resistance as the knee angle nears full extension, thus preventing a terminal impact or in other such determined times during the gait cycle.

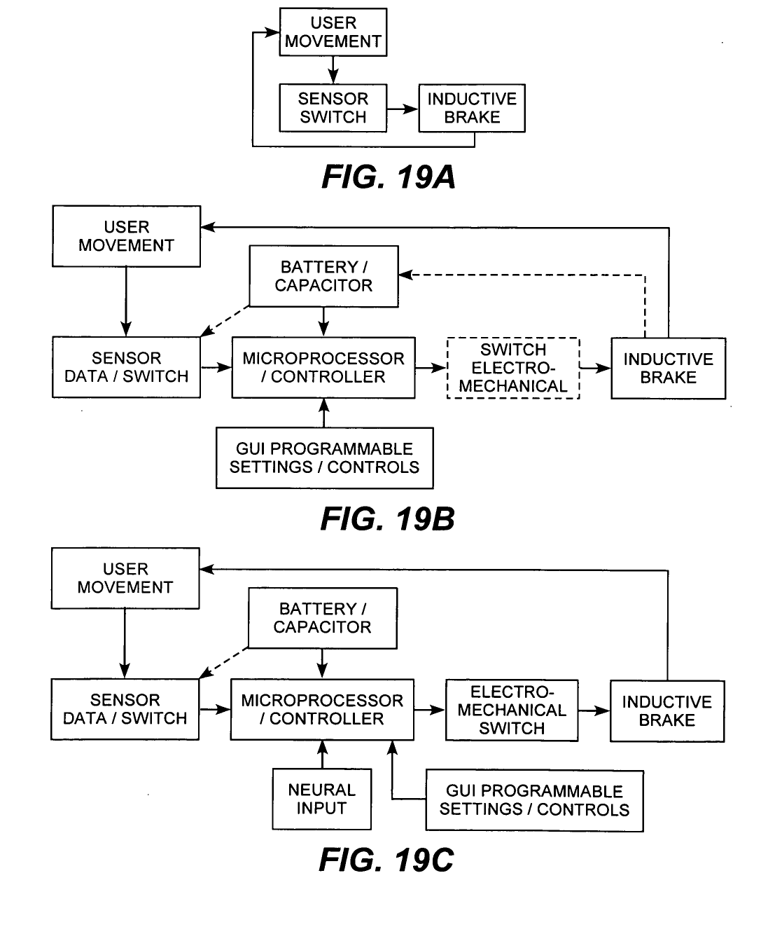

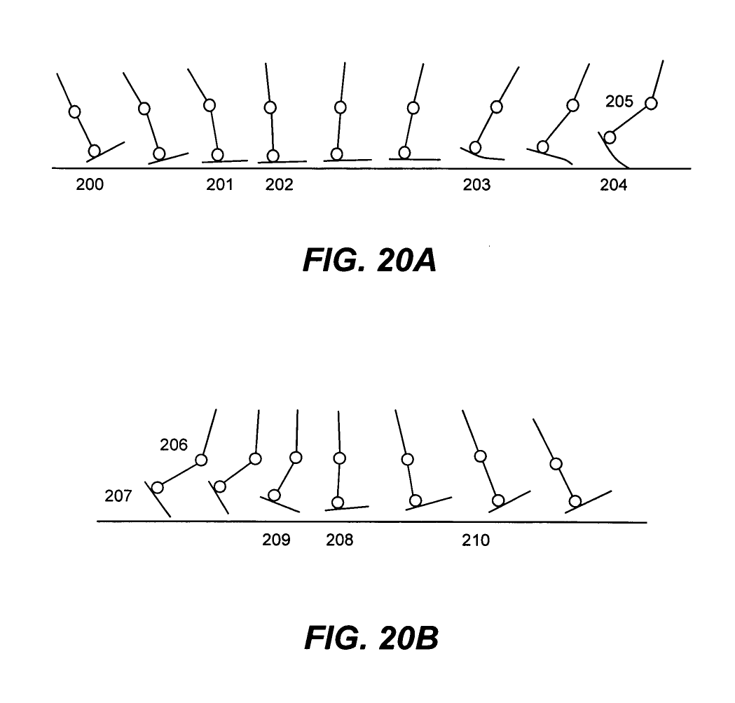

Similar to FIGS. 18, FIG. 19A illustrates the use of a potentiometer, or similar sensor switch, to control the movement of the inductive brake. As the potentiometer may be moved through its tunable range, such as may occur in correlation to the user's joint's position as they may be linked directly or indirectly together (such as in FIGS. 18A, 18B, and 18C), the resistance level change of the potentiometer may provide correlation of inductive braking with the joint angle. This is advantageous to not use a battery or capacitor to power the system, but rather to allow for the electronics circuitry itself to alter the resistance settings, in correlation with joint angular change. Further electronics may be included as well to add function for particular instances in the gait cycle to be determined by linkages to potentiometers or other such sensing devices of the ambulated environment. By way of example, the use of a secondary potentiometer may be used to determine that the knee joint may be nearing terminal impact during swing phase of gait may be used to damped the joint at that moment. Other such electronics may be further incorporated to create additional adaptability of the design to ambulation.

Control of such a system may include a direct link of joint angular position and/or speed with resistance setting. As angular velocity of the joint increases, sensor mechanism may result in increased resistance and hence increased inductive braking. Similarly, as angle of the joint may move through its range of motion, sensor mechanism, such as but not limited to a potentiometer, may increase resistance in the circuit and hence increase the inductive braking of the joint.

It is contemplated that a powered electronic control method could use circuitry powered by a battery, capacitor, or other energy storage device to control the resistive forces of the inductive brake. The battery, capacitor, etc could be charged and stored by the motion of the inductive brake itself, or by any other means of storing electric energy. Conversely, the system may use a long life battery or other battery mechanism to power the sensor and/or microprocessor without the need for recharging, as those subcomponents may require extremely low levels of power. Conversely, the system may be rechargeable.

It is understood that the microprocessor or controller may require power, but only for the purposes of transferring stored control methodology to function the timing and amount of resistance to the joint during ambulation. It is understood that in this embodiment, the joint does not require power from the battery for movement through any mechanical means, but rather simply allows the microprocessor or switching mechanism to turn the inductive brake on and off electronically. Other joints found in the prior art typically rely on electric motors, for instance, for the case of hydraulically controlled systems, to open and close a valve mechanism. This approach results in significantly larger power draw, and therefore is a less desirable method of application for clinical use.

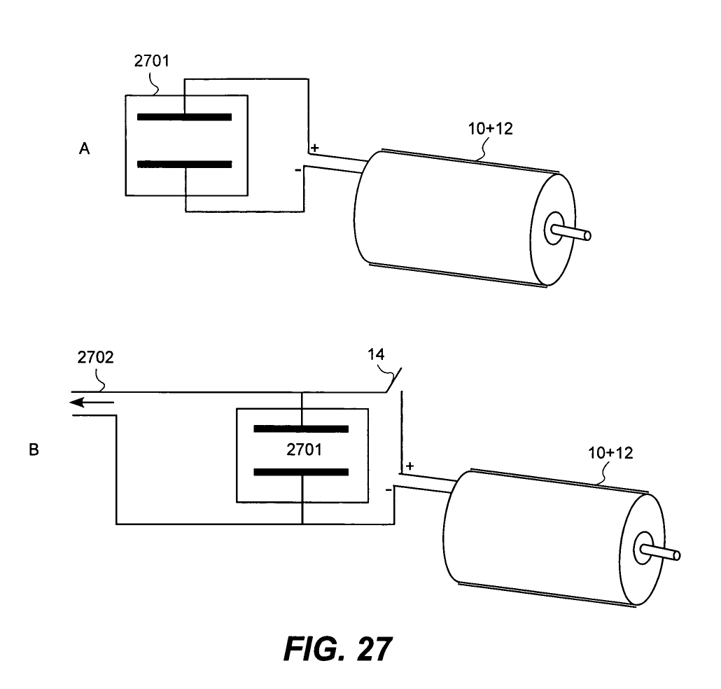

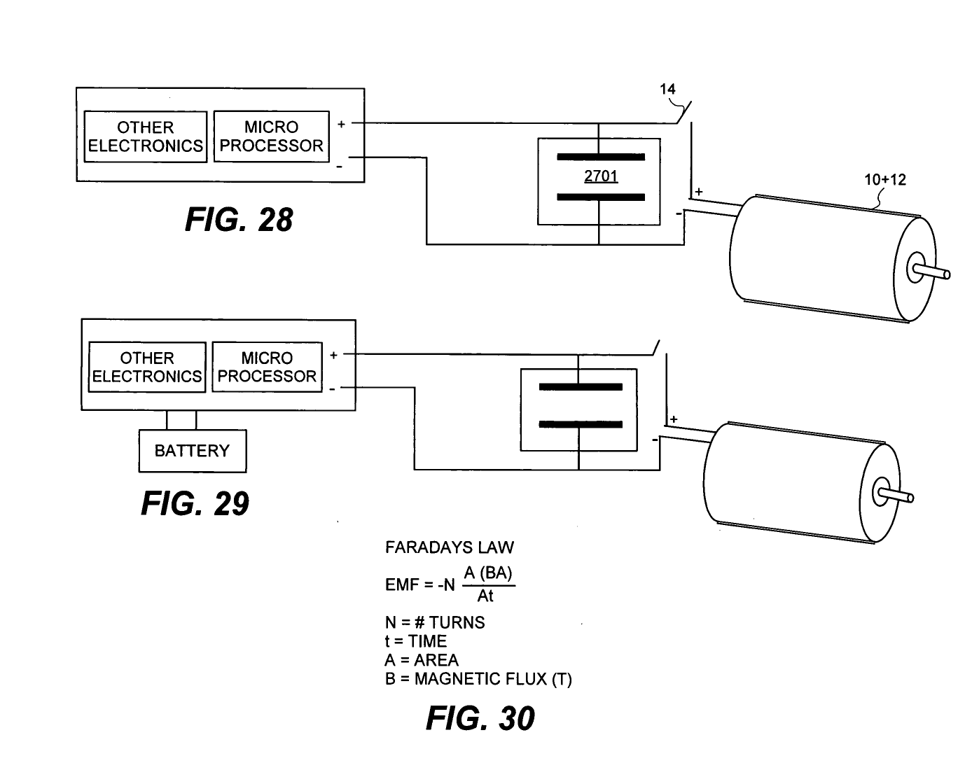

FIG. 27 illustrates on embodiment where energy from the inductive brake may be stored and delivered through a capacity, where capacitor 2701 is placed within a circuit, and may store energy when the inductive brake is in its “on” position, 14 closed, and may later release the stored energy 2702 at a time when 14 causes inductive brake to be in the “off” position shown in B, or in an “on” state, not shown in the figure. This may allow for sensor(s) and/or microprocessor(s) or equivalent to be operated effectively. FIG. 28 further illustrates this embodiment, where capacitor 2701 provides electric power to microprocessor or equivalent, as well as necessary other electronics, which may include sensors.

It is further contemplated, as illustrated in FIG. 29, that partial power may be provided by the capacitor or charging mechanism to the microprocessor and/or electronics. The battery may provide partial power to the same system when capable, necessary, or practical.

The timing of the inductive brake to store such energy would be conjunction with the motion of inductive braking—discussed further in the control section. This would not be reliant on when it may be most optimal for energy to be stored, such as in Donelan patent, where “mutualistic” conditions are necessary, but rather would occur when the ambulatory conditions require conductive braking for optimizing the biomechanics of the prosthesis or orthosis. This, biomechanically, would naturally occur at distinctive times during the gait cycle than Donelan's disclosure.

The inductive braking system may instead be directly linked to the microprocessor and/or sensors as needed during ambulation, without the use of a power storage device such as a capacitor or battery. In such a case, the microprocessor, or similar such device, along with necessary sensors, may take power generated from the inductive brake to power those devices in real-time as the system is in use. Otherwise, the sensors and microprocessor would not be receiving power, and hence not be in an “on” state while the joint is not in motion. By way of example, as illustrated in FIG. 19B, the microprocessor or controller, along with sensors, may or may not need to be charged by a battery or other capacitor device. Initializing the inductive brake mechanism may occur directly through the microcontroller, or through an additional electromechanical or other such switch mechanism. It is understood to those skilled in the art of electronics that there are a number of methods of initializing such a device through a microprocessor, and the examples provided should not be considered limiting.

FIG. 19C further describes the relationship between the user's neural input to the control of the inductive brake mechanism. Methods of neural input may include any known approach for capturing intended movement of the user or types not yet invented. Currently available neural input approaches may include, but are not limited to, superficial or implanted EMG data capture, pattern recognition, cortical or peripheral nerve implants, or other known methods.

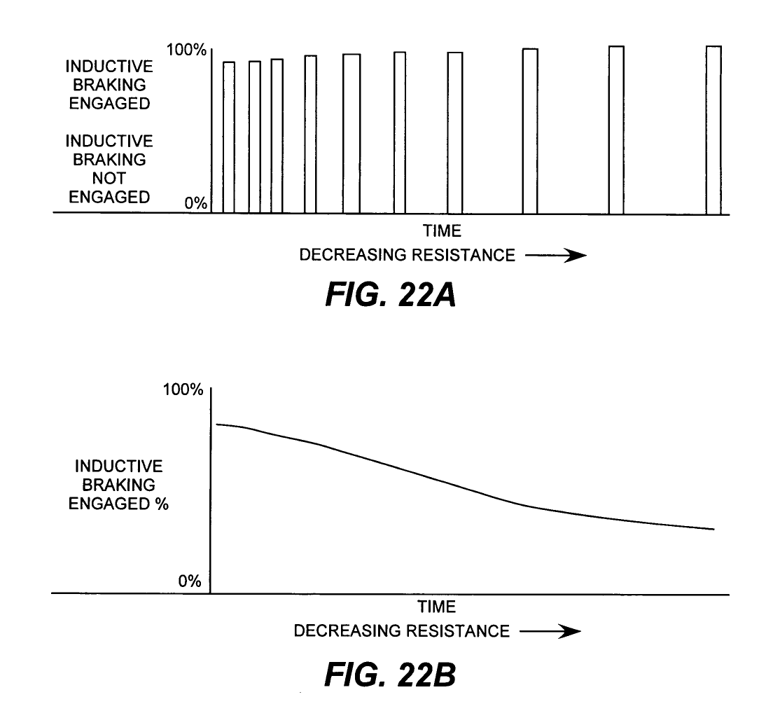

In such examples, the sensor, such as but not limited to a potentiometer, may be the mechanism that induces the control of the inductive brake, or a microprocessor or similar type of device may control the inductive braking. Through the use of a microprocessor, the inductive brake may be controlled using pulse width modulation, as is illustrated in FIG. 22, where as the cycles of the modulation become fewer, the experienced resistance decreases. Conversely, the microprocessor may control the amount of resistance directly. In either case, the overall amount of resistance experienced by the user may be in direct correlation to a set parameter, or may be in proportional control of a set parameter.

The electronic circuitry could take sensor input such as, but not limited to the following: position angle of the joint and/or prosthesis, speed of joint movement and/or prosthesis, weight going through the joint and/or prosthesis, acceleration of the joint and/or prosthesis, angle of the walking terrain, gait speed, etc, and electronically process that information to properly control the “inductive brake” of the joint. It is understood that the term prosthesis and orthosis are used interchangeably for the purposes of simplicity.

It is contemplated that a microprocessor control could be a more sophisticated way of controlling the inductive brake that incorporates the various inputs and may use software and/or algorithms to manipulate the desired output to properly control the inductive brake.

It is also contemplated that the control strategies could be adjusted by the manufacturer, prosthetist, and or patient. It is contemplated, but not limited to, mechanically altering the switch, sensor, etc, pre-compressing a spring on a weight activated switch to adjust the sensitivity of the switch and to compensate for the individual weight of the patient, electronically adjusting the switch, sensor, etc. By example, but not to be considered to limit the invention, it is contemplated to use ‘trim-pots’ to adjust the sensitivity of a sensor, whether or not it is using non-powered electronic or powered electronic strategies, or simply adjusting a switch, and or buttons, etc, to switch into a different mode that has different joint control characteristics such as ‘down-hill skiing mode’, and so forth. It is still further contemplated to using a remote control to achieve any change and or adjusting software that achieves any change. The software may be but is not limited to software on a PC, cell-phone, palm-pilot, and so forth, that may be used to adjust settings, modes, and so on.

The control of the joint may further be adjusted through the use of a graphic user interface. This interface may be used to tailor the dynamic characteristics of the joint to the particular user. By way of example, one user may prefer having high resistance between heel strike and foot flat, while another individual may prefer low resistance. The correlation between the moment in the gait cycle, joint angle, angular velocity, or other such parameters with the resistance of the joint may be customizable by the user or by the practitioner.

It is contemplated that multiple inductive braking mechanisms can be used in joints wherein each can utilize different control strategies. If a single inductive braking mechanism is used, it can also use different control strategies. For example, but not limited to, a joint may use a microprocessor, but if the battery ‘dies’, it can switch to non-powered electronic control. This may enable for a broader or more precise control capability through the use of a powered microprocessor and sensors, though if and when the power storage device may no longer be capable of providing power to the device, the system may switch to a backup mode, where full control may occur through non powered circuitry, such as in illustrated example in FIG. 19A. This example should not be considered limiting, as there may be many added electronics mechanisms to further expand the biomechanics of the system in correlation to the user through more complex circuit design.

The joint and/or prosthesis may also have circuitry of its own for purposes other than inductive braking, such as, but not limited to, EMG input, powered actuation, gait analysis, etc. These may be separate and/or combined with the inductive circuitry.

Any known type of sensor or combination of sensors may be used to assess angle, resistance, forces applied or other information including but not limited to strain gauge, potentiometer, mechanical sensor, electronic sensor, accelerometer, dynometer, pedometer, inclinometer, hall effect sensor, current meter, or any other known method of achieving the same. It is also understood that the same system that uses Lenz's law to resist motion, can also be used to provide active movement of the joint.

While the device may incorporate any number of sensors, switching mechanisms, gearing or equivalent devices, motors or equivalent devices, microprocessors or equivalent devices, or other such mechanisms or components necessary or desirable to induce inductive braking, the control methodology is a vital element to enable the system as a whole to function in accordance with the prosthetic or orthotic user subject. In either the use of this disclosed system for prosthetics or orthotics applications, there is inherently a limitation in the user's functional ambulatory abilities, requiring the disclosed system to enable for full reclaim of ambulatory function. The control methodology of the device is therefore a vital element, to enable it to both function appropriately in accordance with the ambulated environment (hills, stairs, varied speeds, varied loads, etc), but to also function in accordance with the user's own biomechanics (in conjunction with their sound limb for instance, in a prosthetics use).

As illustrated in FIG. 20A, the stance phase of the gait cycle encompasses many segmented instances. Upon heel strike 200, the ankle begins to plantarflex, and the knee experiences slight flexion moment. As the foot moves into foot flat 201 the ankle direction changes to dorsiflexion. Soon after that, the knee flexion moment changes to an extension moment 202, allowing the post heel strike stance flexion to end, and increase in knee stability. Throughout midstance, there is an increasing dorsiflexion moment at the ankle, causing the need for increased resistance during this period of the gait cycle. Toward the end of midstance the loading of the ankle and foot result in slight dorsiflexion to occur 203. At terminal stance, the loading of the foot and ankle spring back 204, resulting in increased plantarflexion—though in the anatomical biomechanics concentric muscular contractions further induce plantarflexion in this stage. At the end of terminal stance and at the beginning of pre-swing, the knee begins to flex 205.

During the swing phase of gait, FIG. 20B, the knee begins moving in a flexion direction 206, and the ankle joint begins moving in a dorsiflexion direction 207. The dorsiflexion at the ankle allows for greater ground clearance during midswing 208. As the thigh section of the limb gains momentum with hip flexion, the knee movement changes direction 209 and begins extending, resulting in full extension by the end of swing. At the end of swing phase of gait however, muscular contractions result in damping of the knee extension so that terminal impact is softened 210.

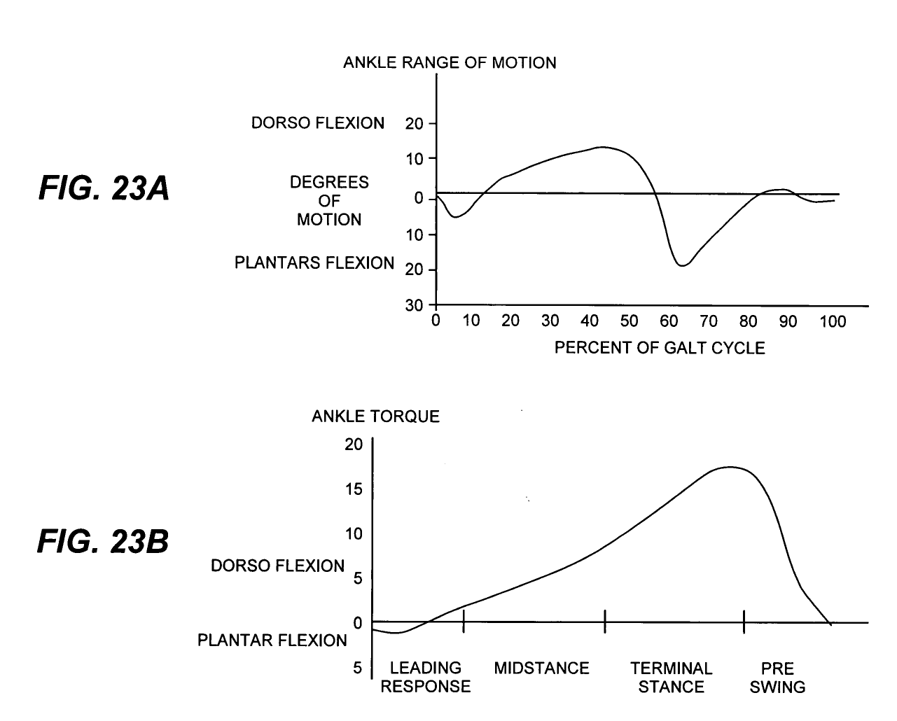

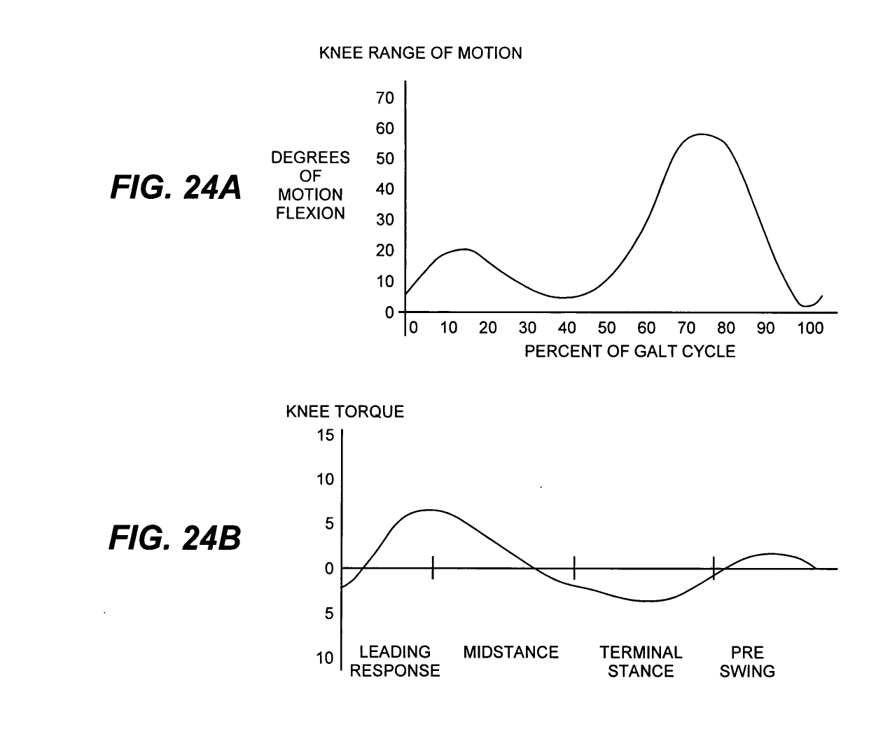

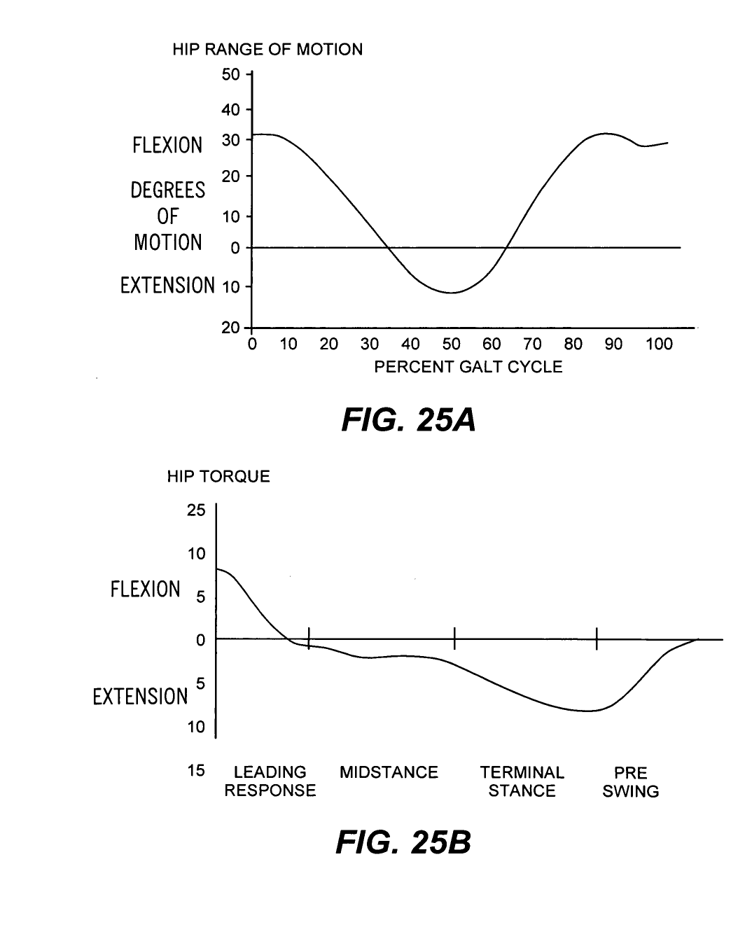

As is further observed in FIGS. 23A, 23B, 24A, 24B, 25A, and 25B, the anatomical ankle, knee, and hip joints all experience complex combinations of range of motion with varied torques. These biomechanically are controlled by concentric and essentric muscular contractions that are very specifically timed in accordance with optimizing energy management and efficiency of the gait cycle, along with accommodation of experienced environmental factors, such as ambulation on varied terrain, at varied speeds, and with varied amounts of forces/loads. The similar control of the orthotic or prosthetic device must use intelligent control to account for these same factors, giving the device the ability to optimize energy efficiency of the gait cycle.

The control methodology described should not be considered limiting, as there are any number of combinations and methods of implementation of replicating and integrating the movement of the device with the anatomical counterpart. The examples provided are meant so that those skilled in the art can appreciate the general example of approach for integrating a prosthesis or orthosis with the human movement.

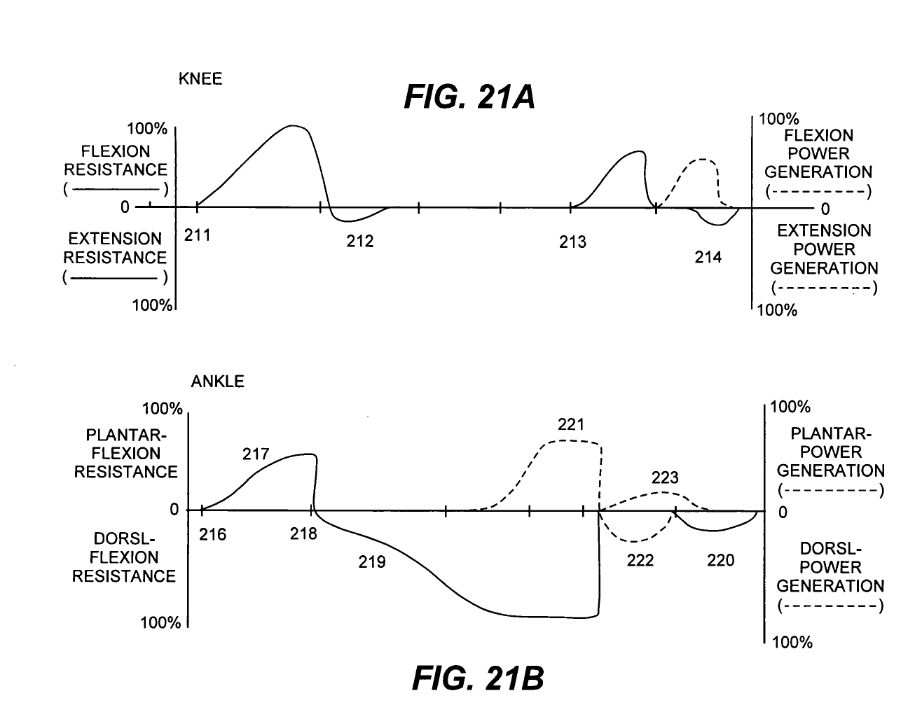

Now generally referring to FIG. 21A and the other illustrations, it is contemplated that the inductive brake knee resistance may function in accordance with a combination of the ambulated environment from sensor data with the nature of typical biomechanics of the user. As illustrated in typical ambulation, the knee resistance increases 211 as post heel strike stance flexion occurs, to limit the amount of stance flexion speed and angle. As the knee direction changes from flexion to extension, the resistance in the extension direction increases slightly 212 in order to allow for the prevention of terminal impact in full extension. This provides a more smooth transition of the knee motion, in symmetry with the user's sound side. Toward terminal stance 213, the flexion resistance of the inductive brake increases slightly in order to limit the amount of heel rise of the shank section of the limb. At the appropriate time, angle, and/or force, the extension resistance may increase 214 in order to limit the amount of terminal impact of the knee as the limb becomes fully extended.

In a case where the inductive brake may be instead powered through applying electric current to it, the system may allow for power generation 215 to allow for spatial orientation of the joint or moved to power the user into a position. In either case, the ability to position the limb in a particular orientation may further help to provide decreased energy expenditure of the user, increased safety, and increased life-like appearance of the motion of the joint. Other moments in the gait cycle may as well induce powered generation of limb movement or spatial orientation in accordance with the ambulated environment. For instance, if the microprocessor or equivalent device determines that the knee is not at appropriate extension toward terminal swing, it may provide further powered extension to allow for the knee have the proper angle at the time of heel strike. Other examples may be drawn as well, illustrating the need for sensors and microprocessor, and overall intelligent control functions of such a system.

The inductive brake ankle joint as well may be controlled dynamically through intelligent control methodology. Upon heel strike 216, the resistance may begin to increase 217 in order to damped the ankle joint prior to foot flat to prevent foot slap. Once foot flat occurs 218, the dorsiflexion resistance increases 219 in order to limit the dorsiflexion moment from causing excessive dorsiflexion of the ankle joint. This may be maintained through toe off. During the swing phase of gait, the dorsiflexion resistance may increase 220 in order to limit the amount of dorsiflexion of the joint, allowing for increased ground clearance, but maintaining appropriate biomechanical symmetry.

In a case where the inductive brake may be instead powered through applying electric current to it, the system may allow for power generation. This may be used during terminal stance to provide active push off of the ankle joint 221—increased plantarflexion. It may also be used to actively dorsiflex the ankle joint during the swing phase of gait 222, otherwise the dorsiflexion may occur through other mechanical means such as but not limited to springs, pulleys, tensioners, or the like. Conversely, the ankle joint may be plantarflexed during the swing phase of gait 223 in accordance with sensor data determining the user may be going down stairs for instance, resulting in a need or desire for increased plantarflexion of the ankle joint prior to contact with the step below.

Similar and comparable methodologies may be used at the hip joint, or other joints, as is illustrated for the knee and ankle joints. In any case, the joints resistance is purposely provided at key times and in key ways to enable for proper biomechanical symmetry. This includes, but is not limited to, angle, angular velocity, resistance to angular change, and others.

In a preferred embodiment, sensor data is provided to microprocessor or similar device, which factors in gait algorithms to determine appropriate resistance of the inductive brake. The algorithms may determine state of gait or other such environmental factors, and may factor in GUI programmable or dial tunable settings in order to provide the correct or determined resistance.

The inductive brake may be operated using pulse width modulation or equivalent function, as illustrated in FIG. 22A. In such a case, the inductive braking may occur at altering frequencies suitable to provide a fluid feeling of altering resistance. An advantage of this type of actuator for prosthetics or orthotics design is that it has an immediate response, with no time delay for motor valve actuated systems for instance.

Alternatively, the inductive brake may operate using a variable resistance setting, such as in the example of changing the dial of a potentiometer, as discussed in FIG. 18.

Each of these moments described are general representations of how the resistance settings of the inductive brake may operate in conjunction with a typical gait cycle of a prosthetic or orthotic user. It should be understood that the figures are not necessarily to scale. They may vary from user to user, may be GUI or dial or set or programmed in any number of ways to tailor the exact nature of the dynamic motion to the user's needs or preferences. The resistance settings may further be altered from step to step in accordance with algorithms that allow for further accommodation and tailoring in real-time to the user's ambulated environment.

While the invention has been described with a certain degree of particularity, it is manifest that many changes may be made in the details of construction and the arrangement of components without departing from the spirit and scope of this disclosure. It is understood that the invention is not limited to the embodiments set forth herein for purposes of exemplification, but is to be limited only by the scope of the attached claim or claims, including the full range of equivalency to which each element thereof is entitled.

I claim:

1. A system as described above.

2. An apparatus as described above.

3. A passive electro-magnetically damped artificial joint comprising a gearing mechanism and motor in circumferential orientation to the other.

4. The passive electro-magnetically damped artificial joint of claim 3 further comprising an inductive braking mechanism that is being controlled by microprocessor that is powered by a battery, which is not charged by the system.

5. The passive electro-magnetically damped artificial joint of claim 4 further comprising a noise diminishing housing
